(19) United States
(12) Patent Application Publication
(10) Pub. No.: US 2002/0077576 A1
(43) Pub. Date: Jun. 20, 2002

(54) Ankle brace

(76) Inventor: David Saraceni, Greenlawn, NY (US)

Correspondence Address: Martin S. Glass, Esq., Suite 200, 300 Vanderbilt Motor Parkway, Hauppauge, NY, 11788, US

(21) Appl. No.: 09/737,133

(22) Filed: Dec. 14, 2000

(51) Int. Cl. A61F 5/00

(52) U.S. Cl. 602/27; 602/23

(57) ABSTRACT

An apparatus is provided that is an ankle brace, having a cover around a user's outer ankle, a securing means securing the cover to the user's foot, at least one tightening strap attaching to the cover and securing the cover to the user's foot, and a connector attaching the securing means to the tightening strap. The cover may be a pliable material such as moldable plastic or a heat hardening gel encasing the entire ankle or a C-shaped material wrapping around the ankle. The securing means may be a stirrup around the bottom of the user's foot or a sleeve covering the entire foot.

### BACKGROUND OF THE INVENTION

1. Field of the Invention

This invention relates generally to an ankle brace or splint and more particularly to a device that can be used for protecting, supporting and stabilizing the human ankle from injury, re-injury or during the rehabilitation stages.

2. Description of the Related Art

The ankle is a hinge joint formed by the articulations of the tibia, the malleolus of the fibula and the convex surface of the talus. Ankle injuries are relatively common, particularly as a result of athletic and sports activities.

In more detail, the joint referred to as the ankle consists of two joints which have voluntary movement. They are the talocrural and subtalar joints. The talocrural joint is formed by the lateral malleolus (distal end of the fibula), the medial malleolus (distal end of the tibia), and the talus. The talocrural joint performs dorsiflexion (flexion of the foot) and plantar flexion (pointing of the foot). The subtalar joint is comprised of the talus and the calcaneus. This joint performs motion in two planes. The first set of motions are inversion and eversion. Inversion is inward movement of the sole of the foot. Eversion is the exact opposite movement, turning of the sole of the foot in an outward direction. Adduction and abduction are the second set of movements. Inversion is usually accompanied by adduction (medial flexion) of the anterior part of the foot. Eversion is usually accompanied by abduction (lateral flexion) of the foot. Just above the talocrural joint is the ankle mortise which is also referred to as the distal tibiofibular joint. The distal tibiofibular joint is a syndesmosis joint. A syndesmosis joint is a fibrous articulation in which bony surfaces are bound together by fibrous tissue. There is no significant voluntary movement of this joint. Any significant degree or frequency of motion of this joint is pathological.

There are ligaments on both the medial and lateral sides of the above-identified joints. The strong medial ligament is composed of four parts. As a whole it is referred to as the deltoid ligament. This ligament checks and limits eversion.

There are three separate ligaments on the lateral side of the above-mentioned joints. The lateral ligaments consist of the anterior talofibular, calcanealfibular and posterior talofibular ligaments. These ligaments check and limit inversion or a combination of inversion and plantar flexion. Injury to the lateral ligaments is commonly referred to as a lateral ankle sprain. Frequently ignored ligaments in this region are the distal tibiofibular ligaments. The distal tibiofibular ligaments are referred to as the anterior and posterior tibiofibular ligaments. Recently injury to the distal tibiofibular ligaments has been frequently diagnosed by the medical field as a high (lateral) ankle sprain. Lateral ankle sprains often occur from unpredictable and pathological weight bearing situations.

Although the following list does not include all possibilities, it does describe some of the most common scenarios. These scenarios include taking a step onto an uneven surface where the forefoot lands lower than the rearfoot, landing from a jump on even or uneven surface, stepping onto a object on the ground, simply losing one's balance momentarily or beginning to trip and then attempting to regain his or her balance, thereby disrupting the gait cycle.

In these and numerous other situations, there is excessive force in an inverted subtalar joint position with a neutral or plantarflexed talocrural joint position. These positions exhibit excessive force on the lateral ankle and tibiofibular ligaments. Neutral position is the middle position in between plantarflexion and dorsiflexion. The ligaments passively check and limit these forces. Numerous muscles including the peroneals and anterior tibialis actively check the inversion or inversion and plantar flexion forces, by moving the ankle away from these injury susceptible positions.

If the external forces exhibited on the ankle and lower leg are greater than the body's responding forces, injury occurs. A grade one sprain is defined as injury to the anterior talofibular ligament. A grade two sprain is defined as injury to the anterior talofibular and calcanealfibular ligaments. Finally, injury to the anterior and posterior talofibular and calcanealfibular ligaments is a classified as a grade three ankle sprain.

There are four stages in which soft tissue injuries can be categorized. The stages are the acute, subacute or remodeling stage, the rehabilitation or functional stage and the chronic stage. The acute stage commences immediately after the injury and can last a few hours or up to a few days. During this stage, rest and limited weight bearing provide the optimal environment for healing and reducing the individual's symptoms.

During the subacute stage, the focus changes to progressively returning to daily activities, such as walking and stair climbing. This can be accomplished by numerous methods which may consist of physical therapy, and/or a brace or other medical interventions. Once the rehabilitation phase has begun, the goals are to eliminate all symptoms and return the individual to any and all activities the individual performed prior to the injury. This group of activities may or may not include the etiology of the injury.

The latter two stages are those in which braces that permit mobility are most beneficial. The chronic stage relates to the environment where the ankle ligaments repeatedly are being injured. In response to repeated trauma, those ligaments lose some of their ability to check and limit external forces, therefore causing repetitive damage. As a result, the body begins to adapt to functioning with a constantly injured ankle. This stage is a pathological stage and is not uncommon for ankle joints. It may be due to a multitude of reasons, including poor progression through the other three stages, unreasonable ankle joint forces not attenuated to over time in a originally healthy ankle, insufficient rehabilitation, an inadequate brace joint or capsule and ligament laxity.

Ankle supports and braces are frequently used in an attempt to limit or further check the excessive forces which cause these sprains. In order to accomplish this they passively limit physiological motion in inversion and plantarflexion. Some of these and other braces only compress the ankle joints. Not only is there the possibility of limiting motion in the potentially injurious directions, these braces may even restrict motion in other directions. There may also be a decrease in the speed of movement of any joints involved. This could be counter-productive since muscle contraction is one of the two major mechanisms by which the ankle checks and limits excessive motion. All these mechanisms may require greater energy to perform activities. This may appear a minor side effect, but when it needs to be performed repetitively, as in walking or jogging, or if speed during athletic activities is crucial, performance can be compromised.

Other disadvantages of many of these braces include the lack of adjustments, or customization of the brace to an individual's lower leg and foot. Another disadvantage of numerous braces is that they do not attempt to correct the resulting laxity (increased flexibility) in the injured ligaments occurred during the injury. If an individual continues to function with laxity in these injured ligaments, the laxity may become permanent. This, and other scenarios, including frequently spraining the same ankle, can result in the development of a chronic ankle sprain. The increased laxity of the ligaments and/or instability of the ankle joints can result in the speed, quality and amount of joint mobility may be compromised, and the ability for muscles to function optimally.

Many of these braces are simply making the individual aware of their previous ankle injury, which limits the individual's performance through mental mechanisms. Other disadvantages include the amount of time to put on and remove the brace and the discomfort when wearing them. This is especially true during activities that test the limits of the brace, and the brace being able to fit comfortably in the shoe or sneaker.

The other common method of treating ankle sprains is taping of the ankle frequently in a figure “8” configuration. Medical research has indicated that the effects of this method last only temporarily. This method is also difficult to accomplish alone. Preventive medicine is a rapidly growing aspect of health care. Preventive medicine should address the most common soft tissue injury, the ankle sprain. In order to improve preventive medicine, the remedy needs to affect performance as little as possible. If there is a significant drop in performance when wearing the brace, individuals will stray away from these preventive tactics. Ankle sprains continue to be a major soft tissue injury and this illustrates the lack of success the prior art braces currently demonstrate.

A need therefore exists for an ankle brace that can be used for preventive purposes and throughout all four of the healing stages.

### SUMMARY OF THE INVENTION

In accordance with the principles of the invention, an ankle brace is achieved by having a device that can be wrapped around or molded to an individual's ankle allowing the brace to be used for protecting, supporting and stabilizing the human ankle from injury, re-injury or to be used during the rehabilitation stages.

According to one aspect of the present invention, an ankle brace is achieved by having a pliable material, such as a gel encased in a semi-hardened material, molded to the user's ankle. This material is then firmly strapped in place around the ankle.

Another aspect of the present invention is where a C-brace is wrapped around the ankle to an ankle sleeve and having straps attached to the opposite side of the brace. Both of these aspects of the invention will give the proper stability an individual will need throughout the rehabilitation stages. This occurs by adjusting the amount of compression and force at the distal tibiofibular and/or talocrural joint. The brace may also be useful for individuals who have acute ligament laxity due to a recent ankle sprain.

The brace may internally rotate and/or posteriorly mobilize the fibula. This can negate positive lateral and high ankle sprain tests. It also improves one's balance by placing the distal tibiofibular joint and also the talocrural and subtalar joint, in an improved functioning position. The brace encourages physiological motion and adjusts the bone position prior to all ankle movements. Since its objective is not to limit movement, it may be more readily accepted for high level activities where any slight limitations in movement can affect performance. It will also be more readily accepted by preventive medicine scenarios because of this encouragement of motion.

### DESCRIPTION OF THE DRAWINGS

So that one skilled in the art to which the subject invention appertains will better understand how to practice the present invention, preferred embodiments of the apparatus and method will be described in detail herein below with reference to the drawings wherein:

FIG. 1 is a laid-out view of an ankle brace in accordance with the principles of the present invention;

FIG. 2 is a lateral view of an enhanced ankle brace on the right foot in accordance with the principles of the present invention;

FIG. 3 is a cross-sectional view of a lateral section of the brace at the upper third of the brace in accordance with the principles of the present invention;

FIG. 4 is a side view of an ankle brace shown laid out in accordance with the principles of the present invention;

FIG. 5 is a medial view of an enhanced ankle brace on the left foot in accordance with the principles of the present invention.

FIG. 6 is a lateral view of an alternate embodiment of an ankle brace on the right foot in accordance with the principles of the present invention;

FIG. 7 is an interior lateral view of an alternate embodiment of the ankle brace in accordance with the principles of the present invention; and

FIG. 8 is a medial view of an alternate embodiment of the ankle brace on the left foot in accordance with the principles of the present invention.

### DETAILED DESCRIPTION OF PREFERRED EMBODIMENTS

FIG. 1 shows a first embodiment of a laid-out view of an ankle brace 10 in accordance with the principles of the present invention. In FIG. 1 the body 2 of the brace consists of a flexible material, such as a heat molded rigid plastic, or a semi-pliable material. Once the brace is in place around the user's foot and ankle, it is molded to fit the individual ankle. An elastic material 4 is used to wrap underneath the arch of the user's foot. This elastic material 4 attaches to a padded material 22, such as foam padding or rubber, or a gel encased in a moldable plastic or hardened rubber, underneath the body 2 of the brace. The elastic material 4 connects on the opposite end to either or both the self-gripping material 6 and the padded material 70 on the underside of the self-gripping material 6. The self-gripping material 6 may consist of a self sticking substance or a hook and loop means. The forward self-gripping strap 8 loops through the forward opening 18 of the body 2, and around the forward border strip 14. The rear tightening strap 12 loops through the rear opening 20 of the body 2, and around the rear border strip 16. The very end of the self-gripping strap 8 and rear tightening strap 12 are attached to themselves by, for example, stitching or hook and loop. This forms a closed loop securing the self-gripping strap 8 and the rear tightening strap 12 to the body 2. Underneath the self-gripping material 6 can be a layer of padded material 70 to protect the inside of the ankle from becoming chaffed or irritated.

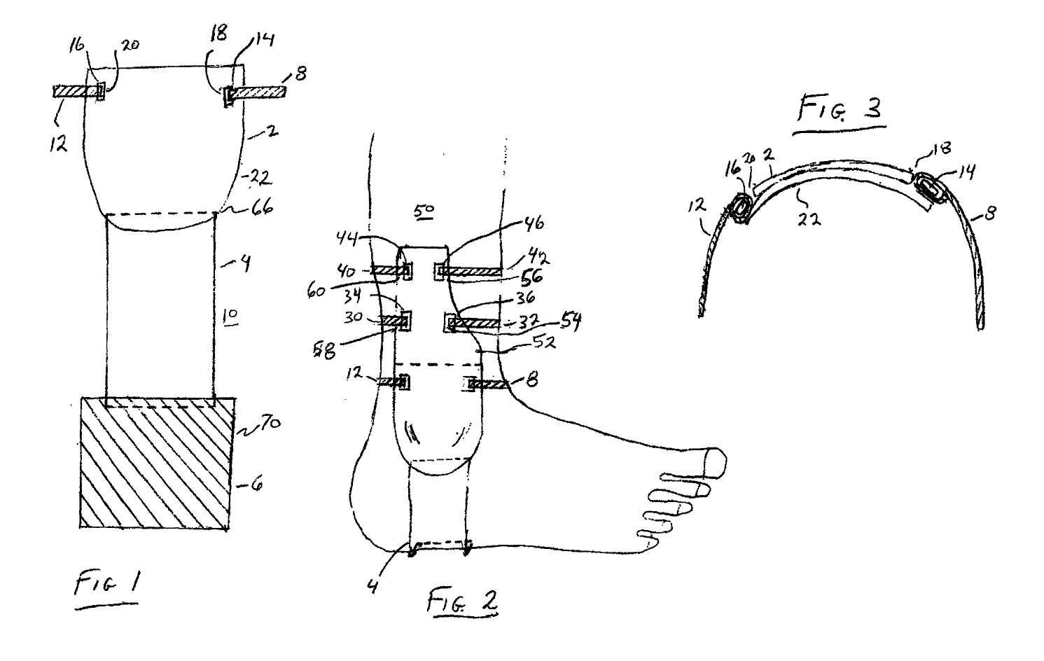

FIG. 2 shows a lateral view of an enhanced ankle brace on the right foot in accordance with the principles of the present invention. This shows an extended ankle brace 50. The self gripping straps 8, 12 wrap around the lower leg and attach to the self gripping material 6. The body 2 grasps the lateral malleolus (distal end of the fibula) and extends superiorly. The elastic material 4 wraps underneath the foot. The self gripping material 6 lies on the medial side of the medial lower leg. The extended body 52 may have a parabolic anterior border. The extended body 52 is a continuation of the body 2 of the first embodiment of FIG. 1. The extended body 52 has two additional sets of forward self-gripping straps 42, 32 and rear tightening straps 40, 30. These self-gripping straps 42, 32 and rear tightening straps 40, 30 pass through the forward openings 46, 54 and rear openings 44, 34, respectively, and wrap back around the forward border strips 56, 36 and rear border strips 60, 58. This is occurs in the same manner described for the self-gripping strap 8 and rear tightening 12 on the typical brace body 2.

To be correctly used, the body 2 of the brace 10 is molded to the user. The elastic material 4 is then secured under the user's foot to prevent the brace from sliding upward. Then the forward self-gripping strap 8 is secured around the user's leg tight enough only so that the body 2 does not move. Finally, the rear tightening strap 12 is then tightened around the user's ankle, forming a rearward pressure on the ankle. The elastic material 4 is not stretched or tightened. It is only placed around the user's arch stopping the brace 10 from moving up the user's leg.

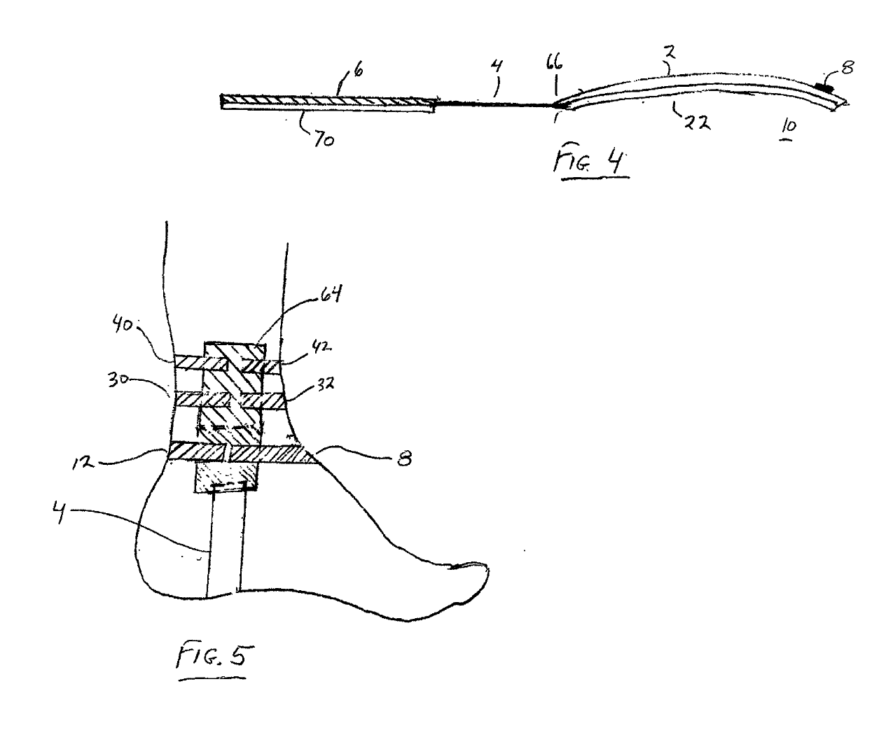

FIG. 3 shows a cross-sectional view of a lateral section of the brace at the upper third of the brace. The padded material 22 is attached to the brace body 2 to be molded around the user's ankle. The self-gripping strap 8 and rear tightening strap 12 are shown wrapping around the outside borders of the openings 14, 16 to the inside borders 18, 20 on the body 2.

FIG. 4 is a laid out side view of an ankle brace 10. The elastic material 4 is attached to the padded material 22 and/or the body 2 at point 66. At the other end the elastic material 4 attaches to the underside of the self-gripping material 6.

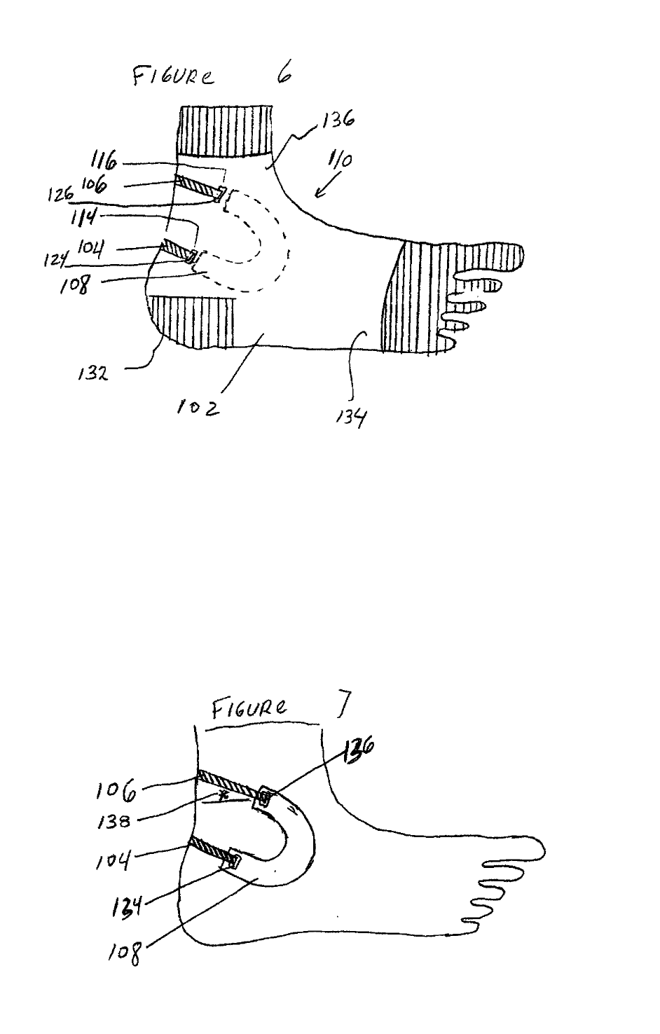

In FIG. 5, an enhanced version of the ankle brace is shown, with the self-gripping straps 42, 32 and rear tightening straps 40, 30 attaching to the extended self-gripping material 64. The extended self-gripping material 64 is a continuation of the self-gripping material 6 in FIG. 1. The outside of the self-gripping material 64 is typically of a hook and loop type. The body 2 is molded to the user's ankle for maximum benefits. The elastic material 4 then is placed around the user's heel and arch to secure the ankle. Next, the self-gripping straps 8 and 42, 32 are secured around the front of the user's ankle. Finally, the rear tightening straps12 and 40, 30 are pulled tautly around the ankle and shin of the user's leg and are secured to the gripping material 64.

An alternate embodiment of the ankle brace 110 is shown in FIG. 6. This is where an ankle sleeve 102 tightly covers the entire ankle and arch of the user's foot. Inside of the ankle sleeve 102 is a C-brace 108 moving and holding the ankle into an adjusted position. The ankle sleeve 102 is typically made of an elastic material such as neoprene. It begins at the distal lower leg 136 and extends proximal to the toes 134, leaving the user's heel 132 exposed. The ankle sleeve 102 may consist of a closed cell sleeve or an open mesh weave. The lower self-gripping strap 104 and upper self-gripping strap 106 exit from the underside of the ankle sleeve 102. The self-gripping straps 104, 106 move through the holes of the elastic material 114, 116, respectively. The self-gripping straps 104, 106, which may consist of a hook and loop means, extend in a posterior or posterior-superior direction. This will depend upon the angle of exit from the ankle sleeve 102. The lower eyelet 124 and upper eyelet 126 around the holes of the elastic material 114, 116 respectively consist of a semi-rigid material covering, such as thick cloth, rubber or plastic. The lower eyelet 124 and upper eyelet 126 are typically rectangular in shape and assist in preventing tears in the ankle sleeve 102. The C-brace 108, shown in dotted lines, is attached on the underside of the brace 110. This attachment can be accomplished in a multitude of methods, including being sewn together or adhered to each other by glue, tape or hook and loop.

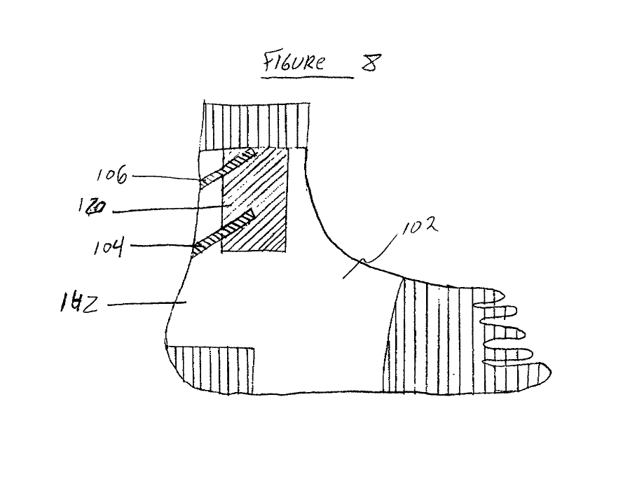

FIG. 7 is a lateral view of the alternate embodiment of the ankle brace 110. For clarity, this is being shown without the ankle sleeve 102. The C-brace 108 grabs around the lateral malleolus of the fibula. The C-brace 108 wraps around the lateral malleolus from the superior/anterior aspect to the inferior/posterior aspect. Although malleable, the C-brace 108 is not stretchable and is typically constructed from a medium to high density foam and/or rubber composite. The self-gripping straps 104, 106 attach to the distal ends of the C-brace 108 by first moving through the holes of the C-brace 134, 136. The self-gripping straps 104, 106 then attach to themselves through the holes in the C-brace 134, 136. The angle 138 of the C-brace 108 and the self-gripping straps 104, 106 can vary from zero to sixty degrees.

FIG. 8 is a medial view of the ankle brace 110. The self-gripping straps 104, 106 move around the back of the lower leg 142 and attach to the self-gripping material 120. Depending upon the angle of the self-gripping straps 104, 106 they will attach in either a posterior or superior/posterior orientation.

The proper order of steps for the user to wear the alternate embodiment of the ankle brace 110 are as follows. First, the lower self-gripping strap 104, and upper self-gripping strap 106 are loosely brought through the lower eyelet 124 and upper eyelet 126. Next, the ankle sleeve 102 is slipped onto the user's foot in the same manner as putting on a sock. Once the ankle sleeve 102 is situated properly around the ankle, the user moves into a standing or at least partially weight-bearing position. The user remains in this position for the remainder of the time to properly place the brace 110. The user then adjusts the placement of the C-brace 108, wrapping it around the lateral malleolus. The self-gripping straps 104, 106 are then pulled around the back of the lower leg. The self-gripping straps 104, 106 can be pulled in any order. Finally, the self-gripping straps 104, 106 are attached to the self-gripping material 120 with firm pressure.

Although the subject invention has been described with respect to preferred embodiments, it will be readily apparent to those having ordinary skill in the art to which it appertains that changes and modifications may be made thereto without departing from the spirit or scope of the subject invention as defined by the appended claims.

What is claimed is:

1. An ankle brace, comprising:
covering means covering a user's outer ankle;
a securing means securing said covering means to said user's foot;
at least one tightening strap attaching to said covering means securing said covering means to said user's foot; and
a connector attaching said securing means to said tightening strap.

2. The ankle brace of claim 1, further comprising at least one forward strap attaching to said covering means securing said covering means to front of said user's foot,
wherein said connector attaches said securing means to said forward strap.

3. The ankle brace of claim 2, wherein said securing means is a stirrup.

4. The ankle brace of claim 2, wherein said covering means is pliable, semi-rigid material, molding to said user's outer ankle.

5. The ankle brace of claim 4, wherein said stirrup is an elastic material wrapping underneath said user's arch.

6. The ankle brace of claim 3, wherein said covering means further comprising a gel encased in a heatable material.

7. The ankle brace of claim 3, wherein said covering means extends above said user's ankle.

8. The ankle brace of claim 7, wherein a second forward strap further attaching to said extended covering means securing said covering means to front of said user's foot.

9. The ankle brace of claim 7, wherein said covering means further comprising a gel encased in a heatable material.

10. The ankle brace of claim 8, wherein a second tightening strap further attaching to said extended covering means securing said covering means to rear of said user's foot.

11. The ankle brace of claim 10, wherein an elongated connector further attaching said stirrup to said at least one forward strap and said second forward strap, and said at least one tightening strap and said second tightening strap.

12. The ankle brace of claim 1, wherein said covering means is a C-shaped, malleable material, wrapping around said user's outer ankle.

13. The ankle brace of claim 12, wherein said securing means further comprises a sleeve attaching to said covering means.

14. The ankle brace of claim 12, wherein said at least one tightening strap further comprises a plurality of tightening straps that moves around the rear of said user's foot at an upward angle from 0 to 60 degrees.

15. An ankle brace, comprising:
covering means covering a user's outer ankle,
wherein said covering means further comprising a gel encased in a heatable material;
a stirrup securing said covering means to bottom of said user's foot;
a plurality of forward straps attaching to said covering means securing said covering means to front of said user's foot;
a plurality of tightening rear straps attaching to said covering means securing said covering means to rear of said user's foot; and
a connector attaching said stirrup to said plurality of forward straps and plurality of tightening rear straps.

16. The method of bracing an ankle comprising the steps of:
wrapping said ankle with a cover;
molding said cover in a rear facing C-shape around said ankle;
securing said cover to a user's foot; and
applying pressure around said ankle towards rear of said user's foot.

17. The method of claim 16, wherein said step of applying pressure farther includes pulling said cover towards rear of user's ankle.

18. The method of claim 16, wherein said step of securing said cover includes sliding said user's foot into an elastic sleeve and securing said cover to said sleeve.

19. The method of claim 18, wherein said step of applying pressure further includes pulling said cover towards rear of user's ankle.

20. The method of claim 19, wherein said step of pulling said cover further includes having said C-shaped cover angling between 0 and 60 degrees.
